(19) United States
(12) Patent Application Publication
(10) Pub. No.: US 2007/0112257 A1
(43) Pub. Date: May 17, 2007

(54) Laryngoscope

(76) Inventor: Fritz Hensler, Neuhausen (DE)

Correspondence Address: ST. ONGE STEWARD JOHNSTON & REENS, LLC, 986 BEDFORD STREET, STAMFORD, CT, 06905-5619, US

(21) Appl. No.: 11/566,077

(22) Filed: Dec. 1, 2006

Related U.S. Application Data
(63) Continuation of application No. PCT/EP05/05623, filed on May 25, 2005.

(30) Foreign Application Priority Data
Jun. 3, 2004 (DE) 10 2004 028 428.8

(51) Int. Cl. A61B 1/267

(52) U.S. Cl. 600/199

(57) ABSTRACT

A laryngoscope has a laryngoscope spatula, a handle, and an illumination device for illuminating the oral and pharyngeal cavity, the illumination device having at least a first light-radiating element, which is arranged on the laryngoscope spatula and, when operating, radiates a cone of light with an angle of beam spread. The illumination device has at least a second light-radiating element, which is likewise arranged on the laryngoscope spatula, and the at least one second light-emitting element radiates a second cone of light with a second angle of beam spread which is greater than the angle of beam spread of the cone of light of the first light-radiating element.

### CROSS-REFERENCE TO RELATED APPLICATION

The present application is a continuation of pending international patent application PCT/EP2005/005623 filed on May 25, 2005 which designates the United States, and which claims priority of German patent application 10 2004 028 428.8 filed on Jun. 3, 2004.

### BACKGROUND OF THE INVENTION

The invention relates to laryngoscopes for examining the oral and/or pharyngeal cavity.

Laryngoscopes are medical instruments with which the larynx can be endoscopically examined. In a laryngoscopic examination, the laryngoscope spatula is introduced into the patient's mouth, and its distal end advanced until it is in the vicinity of the larynx.

When introducing the laryngoscope spatula into the oral and pharyngeal cavity, the attending physician must proceed very carefully so as not to cause injuries in the pharyngeal cavity, particularly the larynx area. The attending physician therefore needs to have a very good view of the inside of the mouth and pharynx. To have a good view of the mouth and pharynx, however, it is necessary for them to be adequately illuminated; laryngoscopes are therefore usually equipped with an illumination device.

Laryngoscopes featuring many different types of illumination devices are known.

In the present invention, a light-radiating element is to be understood as an element from which the light is ultimately radiated into the oral and pharyngeal cavity.

The term “cone of light” is to be understood very generally in regard to the shape of the contour and is not restricted to the mathematical sense of the word cone.

The laryngoscope known from the document WO-A-02/071930 has a light-emitting diode (LED) as the light-radiating element, the LED being arranged on the laryngoscope spatula, though in that document it is considered preferable to arrange the light-emitting diode in the handle and to guide the light emitted by the light-emitting diode to the distal portion of the laryngoscope spatula by means of a fibre-optic light guide and radiate it from there to illuminate the oral and pharyngeal cavity.

The use of light-emitting diodes as light sources or light-radiating elements in laryngoscopes has been made possible by further technical development of light-emitting diodes, white light-emitting diodes especially. In particular, light-emitting diodes which guarantee a high light yield are now available.

In older types of laryngoscopes, incandescent bulbs were used for the illumination device, the bulbs being arranged either in the handle—in which case the light emitted by the incandescent bulb is again guided into the laryngoscope spatula via a fibre-optic light guide and radiated from there—or in the laryngoscope spatula itself, the incandescent bulb being powered by a battery in the handle, and the battery being connected to the incandescent bulb in the laryngoscope spatula by means of electrical wires.

A laryngoscope of the last-mentioned type is disclosed in document DE-A-42 43 790, for example.

Document DE-A-102 13 919 discloses a medical instrument, like a wound retractor, brain spatula, muscle retractor, vaginal speculum and the like for example, which has a working part for keeping the intervention site open, the working part having, distally, an illumination device for illuminating the intervention site. The illumination device of this known instrument also possesses at least one white light-emitting diode, which is connected to a power source outside the working part by means of an electrical connection at least part of which is located inside the working part. The illumination device in this known instrument may preferably consist of several arrays of white light-emitting diodes.

The provision of more than one light-radiating element or means of illumination, such as light-emitting diodes, on the laryngoscope spatula allows the total light intensity to be adjusted according to the number of light-radiating elements. However, this does not necessarily lead to better illumination of the whole of the oral and pharyngeal cavity and thus better orientation and better visibility for the physician. Simply increasing the light intensity can even dazzle the physician while he is examining the larynx.

### SUMMARY OF THE INVENTION

An object of the invention is therefore to develop further a laryngoscope of the type mentioned at the beginning, so that the illumination and thus examination of the oral and pharyngeal cavity during laryngoscopy can be improved.

According to the invention, a laryngoscope is provided, comprising a laryngoscope spatula, a handle, and an illumination device for illuminating an observation cavity in the human or animal body. The illumination device has a first light-radiating element arranged on the laryngoscope spatula and, when operating, radiating a first a cone of light with a first angle of beam spread. The illumination device has at least a second light-radiating element arranged on the laryngoscope spatula, the at least one second light-emitting element radiating a second cone of light with a second angle of beam spread which is greater than the first angle of beam spread of the first cone of light of the first light-radiating element.

The present invention too starts out primarily from the premise that the total light intensity may be increased by increasing the number of light-radiating elements on the laryngoscope spatula. In contrast to the laryngoscopes known from prior art, however, the increase in light intensity in the oral and pharyngeal cavity is distributed more evenly, in that the laryngoscope spatula has at least two light-radiating elements which radiate light with different angles of beam spread. The first light-radiating element, which has a smaller angle of beam spread, improves the depth of illumination, whilst the second light-radiating element, which has a bigger angle of beam spread, improves the width of illumination. Instead of concentrating more light in one axis direction only through the use of more light-radiating elements as in the prior art, the laryngoscope embodiment according to the invention achieves improved illumination in two axis directions, which also prevents the physician's being dazzled by an excessive light intensity in one axis direction. In other words, the illumination device of the laryngoscope according to the invention takes better account of the anatomy of the oral and laryngeal cavity than do the illumination devices of known laryngoscopes.

In a preferred embodiment, the light-radiating elements are arranged in such a way that the cones of light partially overlap each other.

The advantage of this is that there is a light-cone area in which the light-intensity distributions of the two light-radiating elements are in part combined, resulting in a higher total light intensity in the area of overlap. It is expedient and advantageous to have the light-radiating elements arranged in such a way that the area of over-lap lies in a roughly rectilinear extension of the longitudinal axis of the laryngoscope spatula. The light-radiating elements are also preferably arranged next to each other, at roughly the same height as each other in relation to the longitudinal axis of the laryngoscope spatula, in the distal portion of the laryngoscope spatula.

In a further preferred embodiment, a first principal direction of radiation of the first light-radiating element is parallel to a second principal direction of radiation of the second light-radiating element.

If the at least two light-radiating elements are arranged close to each other, the two principal directions of radiation referred to above can virtually coincide even. The particular advantage of this measure in conjunction with the measure mentioned above is that a large area of overlap of the at least two cones of light can be achieved.

In a further preferred embodiment, the second angle of beam spread is greater than the first angle of beam spread by a factor of approximately 1.5 to 4.

The advantage of this is that the at least two cones of light differ markedly in respect of their angle of beam spread, thereby further improving both the depth of illumination and the width of illumination.

In a preferred practical embodiment, the first angle of beam spread ranges from roughly 10° to roughly 30° and the second angle of beam spread ranges from roughly 40° to roughly 70°.

In a preferred example, the first angle of beam spread can be roughly 25° for illumination of depth and the second angle of beam spread can be roughly 60° for illumination of width.

In a further preferred embodiment, the first light-radiating element and the at least one second light-radiating element are light sources.

Although it is also possible for the first light-radiating element and the second light-radiating element in the invention to be the exit faces of fibre-optic light guides, the embodiment in which the at least two light-radiating elements are light sources has the advantage that transmission losses resulting from the use of fairly long fibre-optic light guides are avoided. This means that the light is radiated from the light sources, which are preferably arranged in the distal portion of the laryngoscope spatula, directly into the oral and pharyngeal cavity without being transmitted via a fibre-optic light guide.

In a particularly preferred embodiment, the first light-radiating element and the at least one second light-radiating element each comprise at least one light-emitting diode, in particular a white light-emitting diode.

Light-emitting diodes that radiate cones of light with different angles of beam spread are now available and are therefore particularly suitable for use in this invention, especially as such light-emitting diodes are inexpensive and, as mentioned at the start, ones that deliver high light intensities can now be obtained. Furthermore, light-emitting diodes of this type can also be very easily incorporated into the laryngoscope spatula, e.g. into a standard incandescent bulb socket of the kind used in the older type of laryngoscope spatulas, in which incandescent bulbs were employed for the illumination device.

It is preferable, in this connection, if the first light-emitting diode and the at least one second light-emitting diode have a common power supply.

The advantage of this is that there is no appreciable increase in the amount of space required to accommodate the power supply in comparison with conventional laryngoscopes that have only one light source.

In a further preferred embodiment, the power supply comprises at least one battery arranged in the handle.

The advantage of using a battery as the power supply for the at least two light-emitting diodes is that the laryngoscope as a whole is independent, i.e. no external power supply and thus no cables which would interfere with the use of the laryngoscope are required.

The at least one first light-emitting diode and the at least one second light-emitting diode may be connected to the common power supply in parallel or in series; in the case of parallel connection, the light-emitting diodes may be provided with suitable voltage-dropping resistors to limit the voltage; in the case of series connection, an electronic drive circuit can be provided to increase the voltage provided by the battery/batteries as appropriate, in order to achieve the avalanche voltage of the at least two light-emitting diodes.

In a further preferred embodiment, there is a battery-voltage monitoring circuit for the at least one battery.

The advantage of this is that a state in which there is no longer sufficient power to operate the at least two light-emitting diodes can be promptly identified.

In particular, in this connection, it is preferable if the battery-voltage monitoring circuit generates a signal if the battery voltage drops below a predetermined minimum level, the signal being generated by at least one of the two light-emitting diodes.

The advantage of this is that at least one of the two light-emitting diodes has the additional function of indicating when the battery voltage is too low, with the advantageous result that no further light-emitting diode needs to be used for the purpose of such indication as in the case of the laryngoscope known from document WO-A-02/071930 for example.

Further advantages and features will be apparent from in the following description and the enclosed drawing.

It goes without saying that the features mentioned above and those still to be explained below can be used not only in the stated combination but also in other combinations or on their own without going beyond the scope of the present invention.

### BRIEF DESCRIPTION OF THE DRAWINGS

Illustrative embodiments of the invention are shown in the drawing and are described in greater detail with reference thereto below. In the drawings:

FIG. 1 shows a lateral view of a laryngoscope;

FIG. 2 shows an enlarged, partially cut-away, view of the laryngoscope spatula of the laryngoscope in FIG. 1;

FIG. 3 shows a partially cut-away, top view of the laryngoscope spatula in FIG. 2;

FIG. 4 shows a block circuit diagram of the laryngoscope in FIG. 1; and

FIG. 5 shows a block circuit diagram of a possible variant of the laryngoscope in FIG. 1 as an alternative to FIG. 4.

### DETAILED DESCRIPTION OF PREFERRED EXAMPLARY EMBODIMENTS

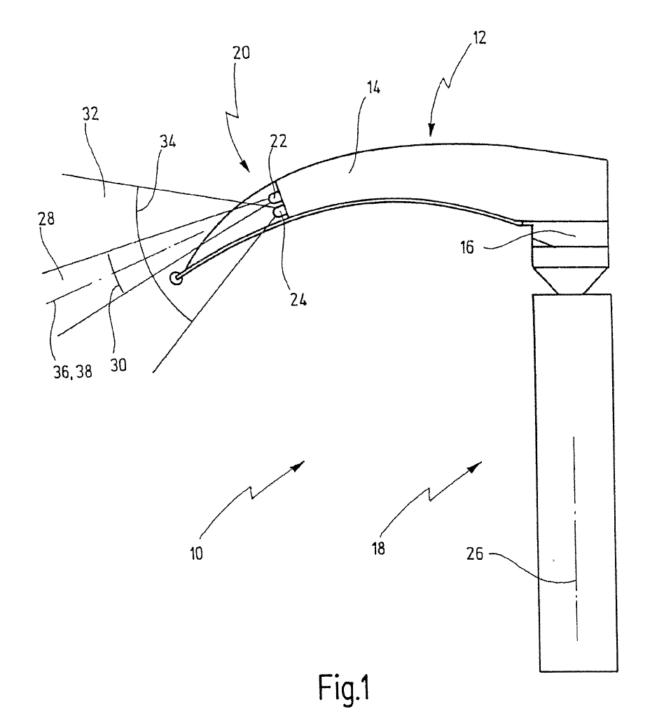

FIG. 1 shows a laryngoscope with the general reference number 10.

The laryngoscope 10 is used in medical and surgical diagnostic procedures, e.g. examination of the larynx.

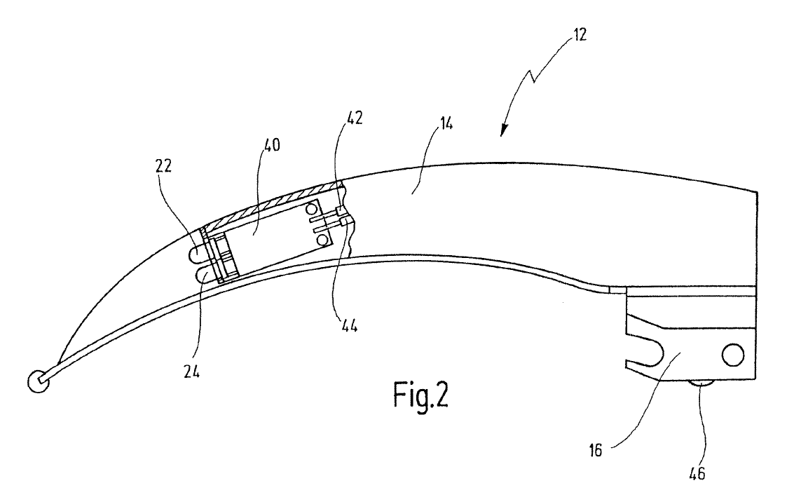

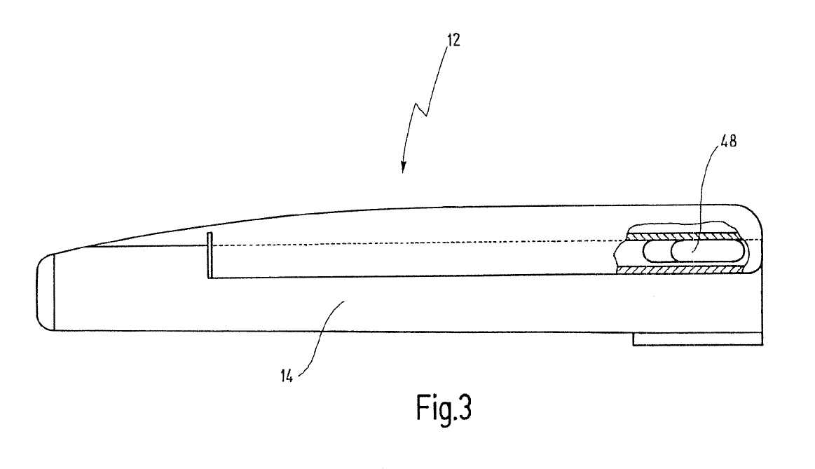

The laryngoscope 10 has a laryngoscope spatula 12, which in FIGS. 2 and 3 is shown in isolation. The laryngoscope spatula 12 has a spatula blade 14 and a spatula head 16. The laryngoscope spatula 12 is detachably connected to a handle 18 via the spatula head 16.

The laryngoscope 10 has an illumination device 20 to illuminate the oral and pharyngeal cavity.

The illumination device 20 has a first light-radiating element 22 and a second light-radiating element 24; further light-radiating elements could be provided.

The first light-radiating element 22 and the second light-radiating element 24 are arranged in the distal portion of the laryngoscope spatula 12, or, to be more precise, the laryngoscope blade 14.

In the illustrative embodiment, the light-radiating elements 22 and 24 are arranged in a row whose orientation follows a handle axis 26, though an arrangement in which they are rotated by 90° on the laryngoscope spatula 12 could be envisaged.

When the illumination device 20 is in operation, the first light-radiating element 22 radiates a first cone of light 28 with a first angle of beam spread 30. The second light-radiating element 24 radiates a second cone of light 32 with a second angle of beam spread 34 when the illumination device 20 is in operation; the second angle of beam spread 34 is greater than the first angle of beam spread 30 of the first cone of light 28.

The light-radiating elements 22 and 24 are arranged close to each other, with the result that the cones of light 28 and 32 partially overlap.

A first principal direction of radiation 36 of the first light-radiating element 22 is parallel to a second principal direction of radiation 38 of the second light-radiating element 24, the two principal directions of radiation 36, 38 completely or virtually coinciding, as illustrated in the drawing in FIG. 1.

In the present illustrative embodiment, the second angle of beam spread 34 of the second cone of light 32 is approximately 60° and the angle of beam spread 30 of the first cone of light 28 is approximately 25°, so the second angle of beam spread 34 is greater than the first angle of beam spread 30 by a factor of about 2.5.

The depth of the oral or pharyngeal cavity can thus be illuminated very well with the first cone of light 28 with the smaller angle of beam spread 30, whilst the width of the oral or pharyngeal cavity can be illuminated very well with the cone of light 32, thereby, overall, giving very good, uniform, illumination of the entire oral or pharyngeal cavity without the laryngoscope 10 needing to be orientated differently for this purpose once it has been introduced into the mouth.

The first light-radiating element 22 and the second light-radiating element 24 are light sources, i.e. light is generated directly in the light-radiating elements 22 and 24.

The light-radiating elements 22 and 24 are light-emitting diodes, white light-emitting diodes especially.

Earlier, it was described how the angle of beam spread 30 of the first light-radiating element 22 is roughly 25°; however, light-emitting diodes that radiate a cone of light with an angle of beam spread ranging from roughly 10° to roughly 30° can also be used. For the second light-radiating element 24, it is also possible to use light-emitting diodes that radiate a cone of light with an angle of beam spread ranging from roughly 40° to roughly 70°.

Specifically, the angles of beam spread 34 and 30 can differ by a factor of 1.5 to 4.

As FIG. 2 shows, the light-radiating elements 22 and 24 in the form of light-emitting diodes are mounted in a socket 40 which is integrated into the spatula blade 14. The socket 40 can be a conventional incandescent bulb socket or a mounting plate to which the light-emitting diodes may then be attached in an appropriate manner.

Two power supply leads 42 and 44 for the positive pole and the negative pole run from the socket 40 to the proximal portion of the laryngoscope spatula 12; the power supply lead 42 (positive pole) is connected to a contact 46 in the spatula head 16.

In the proximal portion of the laryngoscope spatula 12, an appropriate feed-through 48 is provided, as shown in FIG. 3. The power supply leads 42 and 44 are not shown in FIG. 3.

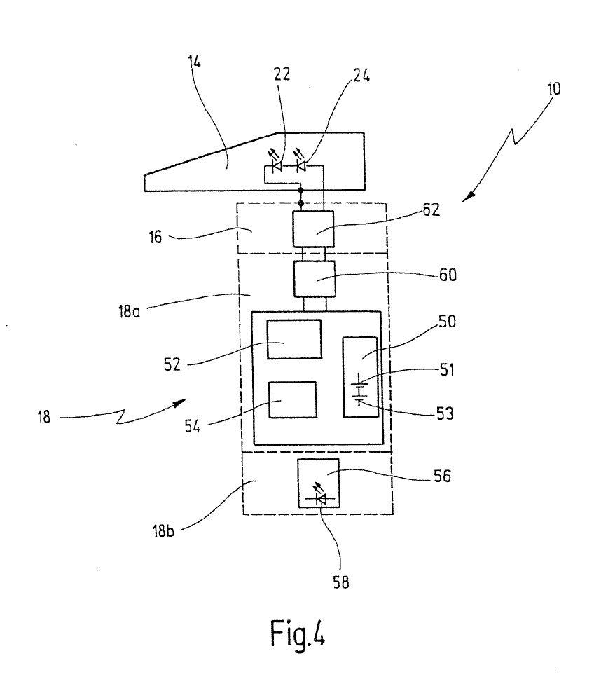

Turning now to FIG. 4, there follows a specific description of the electrical circuit of the light-emitting diodes and the power-supply aspects of the two light-emitting diodes.

FIG. 4 shows a schematic diagram of the spatula blade 14, the spatula head 16, and the handle 18; the figure shows that the handle 18 is divided into a handle main part 18a and a handle end part 18b. The handle end part 18b is formed as a screw cap and can be detached accordingly from the handle main part 18a.

For the light-radiating elements 22 and 24 in the form of light-emitting diodes, there is a common power supply 50, which, in the present illustrative embodiment, comprises two 3-volt batteries 51, 53, delivering a total voltage of 6 V.

As FIG. 4 shows, the power supply 50 is arranged in the handle main part 18a.

In this circuit variant the light-emitting diodes that form the light-radiating elements 22, 24 are connected in series. As a result, the avalanche voltage of the light-emitting diodes together may be somewhat above the 6 V delivered by the power supply.

To ensure that the avalanche voltage of the light-emitting diodes is achieved in this case, an electronic control device in the form of a drive circuit 52, formed as an integrated circuit, is provided in the handle 18.

In addition, the handle contains a battery-voltage monitoring circuit 54 which monitors the voltage of the batteries 51, 53.

The battery-voltage monitoring circuit is connected to a warning device 56, which generates a signal if the battery voltage falls below a predetermined minimum level. Such a signal is for example generated by an additional light-emitting diode 58, which flashes for example if the voltage drops below the predetermined minimum voltage level.

However, the signal can also be generated by one of the two light-radiating elements 22, 24 rather than by an additional light-emitting diode like light-emitting diode 58.

In addition, the handle 18 has an on/off switch 60 and also a top cap connector 62, which forms the electrical connection between the spatula head 16 and the handle 18.

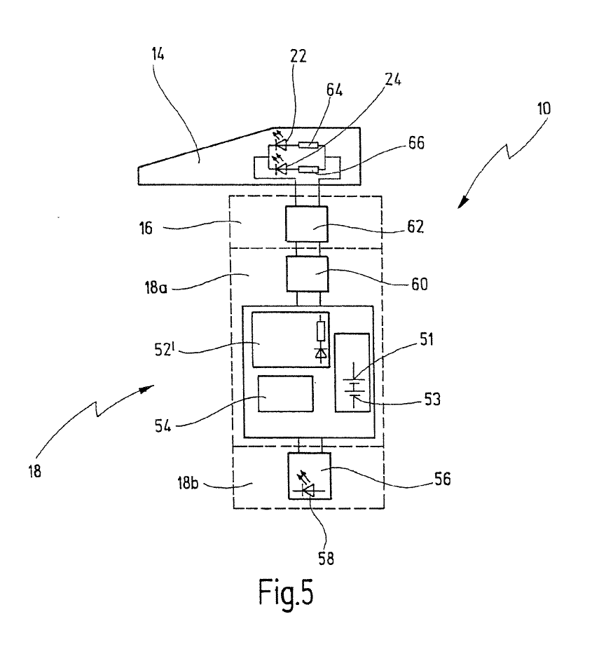

In FIG. 5 the circuit diagram for the two light-radiating elements 22 and 24 in the form of light-emitting diodes is slightly modified compared to that shown in FIG. 4.

In contrast to the circuit variant in FIG. 4, the light-emitting diodes forming the light-radiating elements 22, 24 are connected in parallel. As a result, the avalanche voltage required for the two light-emitting diodes is below the 6 V provided by the batteries 51 and 53. The drive circuit 52 in FIG. 4 is not necessary, therefore. The light-emitting diodes of the light-radiating elements 22, 24 should nevertheless be operated with their own voltage-dropping resistors 64, 66 in order to protect the light-emitting diodes from excessively high currents and to achieve a relatively uniform distribution of light between the two light-emitting diodes.

Here, a voltage-dropping resistor with a diode (reference number 52′) is provided instead of the drive circuit 52 in FIG. 4.

The circuit in FIG. 5 is otherwise the same as the circuit in FIG. 4.

What is claimed is:

1. A laryngoscope, comprising
a laryngoscope spatula,
a handle,
an illumination device for illuminating an observation cavity in the human or animal body,
said illumination device having a first light-radiating element arranged on said laryngoscope spatula and, when operating, radiating a first cone of light with a first angle of beam spread,
said illumination device having at least a second light-radiating element arranged on said laryngoscope spatula, said at least one second light-emitting element radiating a second cone of light with a second angle of beam spread which is greater than said first angle of beam spread of said first cone of light of said first light-radiating element.

2. The laryngoscope of claim 1, wherein said first and at least second light-radiating elements are arranged in such a way that said first and second cones of light partially overlap each other.

3. The laryngoscope of claim 1, wherein a first principal direction of radiation of said first light-radiating element is parallel to a second principal direction of radiation of said second light-radiating element.

4. The laryngoscope of claim 1, wherein said second angle of beam spread is greater than said first angle of beam spread by a factor of approximately 1.5 to 4.

5. The laryngoscope of claim 1, wherein said first angle of beam spread ranges from roughly 10° to roughly 30° and said second angle of beam spread ranges from roughly 40° to roughly 70°.

6. The laryngoscope of claim 1, wherein said first light-radiating element and said at least one second light-radiating element are light sources.

7. The laryngoscope of claim 1, wherein said first light-radiating element and said at least one second light-radiating element each have at least one light-emitting diode.

8. The laryngoscope of claim 7, wherein said at least one light-emitting diode is a white light-emitting diode.

9. The laryngoscope of claim 7, wherein said first light-emitting diode and said at least one second light-emitting diode have a common power supply.

10. The laryngoscope of claim 9, wherein said power supply comprises at least one battery arranged in said handle.

11. The laryngoscope of claim 10, further comprising a battery-voltage monitoring circuit for said at least one battery.

12. The laryngoscope of claim 11, wherein said battery-voltage monitoring circuit generates a signal if the battery voltage drops below a predetermined minimum level, said signal being generated by at least one of said two light-emitting diodes.
